SEQUENCE LISTING (114 sequences)

SEQ ID NO: 1 - DNA, 251 bp, Human
ccagtcgacggccggcggcctgtcaacgtgtacccatgtctgaactggtaccaatcgctggcccgccttccaggtaggaggcgcaaagccatgtaagactacaaatcccagcgtgtaccacgccgtcgccggcagtagagccagctgggagggcgcggagcactatggaaattgtagttccctgctgcggtcccagttacagcgtgaatcccttagcgcaccgcctccccaagtgctgccagcatgctgcc

SEQ ID NO: 2 - DNA, 21 bp, Human
gcggucccaguuacagcgutt

SEQ ID NO: 3 - DNA, 21 bp, Human
ttcgccagggucaaugucgca

SEQ ID NO: 4 - DNA, 21 bp, Human
gggcgcggagcacuauggatt

SEQ ID NO: 5 - DNA, 21 bp, Human
ttcccgcgccucgugauaccu

SEQ ID NO: 6 - DNA, 21 bp, Human
gccggcggccugucaacgutt

SEQ ID NO: 7 - DNA, 21 bp, Human
ttcggccgccggacaguugca

SEQ ID NO: 8 - DNA, 21 bp, Human
ccaaucgcuggcccgccuutt

SEQ ID NO: 9 - DNA, 21 bp, Human
ttgguuagcgaccgggcggaa

SEQ ID NO: 10 - DNA, 21 bp, Human
caggtaggaggcgcaaagctt

SEQ ID NO: 11 - DNA, 21 bp, Human
ttguccauccuccgcguuucg

SEQ ID NO: 12 - DNA, 21 bp, Human
ccaggccuggcaagcacagtt

SEQ ID NO: 13 - DNA, 21 bp, Human
ttgguccggaccguucguguc

SEQ ID NO: 14 - DNA, 21 bp, Human
aucgucagccgugcuagugtt

SEQ ID NO: 15 - DNA, 21 bp, Human
ttuagcagucggcacgaucac

SEQ ID NO: 16 - DNA, 21 bp, Human
aagaccagcaccaggaaugtt

SEQ ID NO: 17 - DNA, 21 bp, Human
ttuucuggucgugguccuuac

SEQ ID NO: 18 - DNA, 21 bp, Human
gcaccgggugccaggagaatt

SEQ ID NO: 19 - DNA, 21 bp, Human
ttcguggcccacgguccucuu

SEQ ID NO: 20 - DNA, 21 bp, Human
cgggugccaggagaacaggtt

SEQ ID NO: 21 - DNA, 21 bp, Human
ttgcccacgguccucuugucc

SEQ ID NO: 22 - DNA, 251 bp, Human
cccagtgaatgtttgttgaattatcagacaaaggaagaaggaacggagcacccggtggtggagacagtgctgggctctgacatgtgtttctctactgcccagatctggaagtcggaatcagcactgtgctgtgaccactcccacccacgctgacttctgtcttgtgtcttcttccaggtctacggctctcgcaggctctgtgagggctttgctatgacctcagtcccctcacggagccacgactgcccctt

SEQ ID NO: 23 - DNA, 21 bp, Human
caggcucugugagggcuuutt

SEQ ID NO: 24 - DNA, 21 bp, Human
ttguccgagacacucccgaaa

SEQ ID NO: 25 - DNA, 21 bp, Human
cgcugacuucugucuugugtt

SEQ ID NO: 26 - DNA, 21 bp, Human
ttgcgacugaagacagaacac

SEQ ID NO: 27 - DNA, 21 bp, Human
cccggugguggagacagugtt

SEQ ID NO: 28 - DNA, 21 bp, Human
ttgggccaccaccucugucac

SEQ ID NO: 29 - DNA, 21 bp, Human
ccaccucacccucccaagutt

SEQ ID NO: 30 - DNA, 21 bp, HUMAN
ttgguggagugggaggguuca

SEQ ID NO: 31 - DNA, 21 bp, HUMAN
ucagccucccagagugcugtt

SEQ ID NO: 32 - DNA, 21 bp, Human
ttagucggagggucucacgac

SEQ ID NO: 33 - DNA, 21 bp, Human
gcuccaguuucccccgcagtt

SEQ ID NO: 34 - DNA, 21 bp, Human
ttcgaggucaaagggggcguc

SEQ ID NO: 35 - DNA, 21 bp, Human
cacuugcugggucccgagutt

SEQ ID NO: 36 - DNA, 21 bp, Human
ttgugaacgacccagggcuca

SEQ ID NO: 37 - DNA, 21 bp, Human
gagacagugcugggcucugtt

SEQ ID NO: 38 - DNA, 21 bp, Human
ttcucugucacgacccgagac

SEQ ID NO: 39 - DNA, 251 bp, Human
tccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttga

SEQ ID NO: 40 - DNA, 21 bp, Human
ggcggauuacuugcccuuatt

SEQ ID NO: 41 - DNA, 21 bp, Human
ttccgccuaaugaacgggaau

SEQ ID NO: 42 - DNA, 21 bp, Human
cctccccaactccatttcctt

SEQ ID NO: 43 - DNA, 21 bp, Human
ttggaggggttgaggtaaagg

SEQ ID NO: 44 - DNA, 21 bp, Human
cacaaugcaggauuccucctt

SEQ ID NO: 45 - DNA, 21 bp, Human
ttguguuacguccuaaggagg

SEQ ID NO: 46 - DNA, 21 bp, Human
gugucucccucguccucugtt

SEQ ID NO: 47 - DNA, 21 bp, Human
ttcacagagggagcaggagac

SEQ ID NO: 48 - DNA, 21 bp, Human
cccccucccguagcuccugtt

SEQ ID NO: 49 - DNA, 21 bp, Human
ttgggggagggcaucgaggac

SEQ ID NO: 50 - DNA, 21 bp, Human
ggcuucagaccugucuccctt

SEQ ID NO: 51 - DNA, 21 bp, Human
ttccgaagucuggacagaggg

SEQ ID NO: 52 - DNA, 21 bp, Human
ccucacagcucuggcuugctt

SEQ ID NO: 53 - DNA, 21 bp, Human
ttggagugucgagaccgaacg

SEQ ID NO: 54 - DNA, 21 bp, Human
acccuagccugccucuccutt

SEQ ID NO: 55 - DNA, 21 bp, Human
ttugggaucggacggagagga

SEQ ID NO: 56 - DNA, 251 bp, Human
gagaggctctaagggacaaggcagggagaagcgcagcggggtgcggggaaccgcacgccctccctttgcctctgcttcccaccccgaggcggcagggcgggcgggcgcggttccgggggtgggcgggctgggcggggcggaggcggggccgcagcactggcttcacccagcctctcccgcccgcagccagagcgagccgagccgcggccagctccggcgggcagggggggcgctggagcgcagcgcagcgc

SEQ ID NO: 57 - DNA, 21 bp, Human
gcgagccgagccgcggccatt

SEQ ID NO: 58 - DNA, 21 bp, Human
ttcgcucggcucggcgccggu

SEQ ID NO: 59 - DNA, 21 bp, Human
uucacccagccucucccgctt

SEQ ID NO: 60 - DNA, 21 bp, Human
ttaagugggucggugugggcg

SEQ ID NO: 61 - DNA, 21 bp, Human
gagaggctctaagggacaatt

SEQ ID NO: 62 - DNA, 21 bp, Human
ttcucuccgagauucccuguu

SEQ ID NO: 63 - DNA, 21 bp, Human
cccugccugcagagcucautt

SEQ ID NO: 64 - DNA, 21 bp, Human
ttgggacggacgtctcgagua

SEQ ID NO: 65 - DNA, 21 bp, Human
gugggcacacguaagugcatt

SEQ ID NO: 66 - DNA, 21 bp, Human
ttcacccgugugcauucacgu

SEQ ID NO: 67 - DNA, 21 bp, Human
ccuaggccucugcccagggtt

SEQ ID NO: 68 - DNA, 21 bp, Human
ttggauccggagacggguccc

SEQ ID NO: 69 - DNA, 21 bp, Human
cccugguccccgggcccactt

SEQ ID NO: 70 - DNA, 21 bp, Human
ttgggaccaggggcccgggug

SEQ ID NO: 71 - DNA, 21 bp, Human
gugcggggaaccgcacgcctt

SEQ ID NO: 72 - DNA, 21 bp, Human
ttcacgccccuuggcgugcgg

SEQ ID NO: 73 - DNA, 20 bp, Human
tgttagaaagctgtctggcc

SEQ ID NO: 74 - DNA, 20 bp, Human
gaggaggcgttgttagaaag

SEQ ID NO: 75 - DNA, 20 bp, Human
tagaggaggaggcgttgtta

SEQ ID NO: 76 - DNA, 20 bp, Human
accggtaattggggtaggga

SEQ ID NO: 77 - DNA, 21 bp, Human
gtgccaactccagagtttcag

SEQ ID NO: 78 - DNA, 20 bp, Human
ggccagacagctttctaaca

SEQ ID NO: 79 - DNA, 20 bp, Human
ctttctaacaacgcctcctc

SEQ ID NO: 80 - DNA, 20 bp, Human
taacaacgcctcctcctcta

SEQ ID NO: 81 - DNA, 20 bp, Human
tccctaccccaattaccggt

SEQ ID NO: 82 - DNA, 20 bp, Human
ctgaaactctggagttggca

SEQ ID NO: 83 - DNA, 1083 bp, Human
  misc_feature (22)..(22): n is a, c, g, t or u
gggatgacgtcgcacccggaantaaagcsgctccgtgacggagcggcggtgcgcgcggcagggcccggagtatcccgctttctttggaggaaacaaccgcatcagatctgcgctgcggcagaggcaggcaagtccctagcgtggaggggcagcatgctggcagcacttggggaggcggtgcgctaagggattcacgctgtaactgggaccgcagcagggaactacaatttccatagtgctccgcgccctcccagctggctctactgccggcgacggcgtggtacactgggatttgtagtcttacatggctttgcgcatcctacctggaaggcgggccagcgattggtaccagttcagacatgggtacacgttgacaggccgccggcgtcgactggcatgttgtgaccattcctggtgctggtcttggtactgttctttcctaccataacttattggaagagggtggcattcctgccttgcagccttttctccagtgaggagtgaacagtgggcacctgagatcctggcccacgctactatgctttcaggctacaaccactagcacggctgacgatggccctttctgcggagaccgagtcacacatctaccgagctctgcgtactgcttctggcgctgccgcccaccttgtggccctgggctttaccatctttgtggctgtgcttgccaggcctggctccagcctgttctcctggcacccggtgcttatgtctttggctttctccttcctgatgaccgaggcactactggtgttttctcctgagagttcgctgctgcactccctctcacggaaaggccgagcacgctgccactgggtgctgcagctgctggccctgctgtgtgcactgctgggcctcggccttgtcatcctccacaaagagcagcttggcaaacccacctggttacgcggcatgggcaggcagggykctggctgttctgtgggcarggctgcaktctcaggtggggtggggcksycyacccmagctgykcccsatggcccctgcgaatcaaastwwccatgtacytytrgggtgkgggyacybctgggaatccaccyyttctgg

SEQ ID NO: 84 - DNA, 19 bp, Human
gcggtcccagttacagcgt

SEQ ID NO: 85 - DNA, 19 bp, Human
gggcgcggagcactatgga

SEQ ID NO: 86 - DNA, 19 bp, Human
gccggcggcctgtcaacgt

SEQ ID NO: 87 - DNA, 19 bp, Human
ccaatcgctggcccgcctt

SEQ ID NO: 88 - DNA, 19 bp, Human
caggtaggaggcgcaaagc

SEQ ID NO: 89 - DNA, 19 bp, Human
ccaggcctggcaagcacag

SEQ ID NO: 90 - DNA, 19 bp, Human
atcgtcagccgtgctagtg

SEQ ID NO: 91 - DNA, 19 bp, Human
aagaccagcaccaggaatg

SEQ ID NO: 92 - DNA, 19 bp, Human
gcaccgggtgccaggagaa

SEQ ID NO: 93 - DNA, 19 bp, Human
cgggtgccaggagaacagg

SEQ ID NO: 94 - DNA, 3479 bp, Human
gctgatggaagggaggtcagcccacagcctggctgggccttggtcatctggcttccggcttcatgatttaatggctcacttgggaaactgaaatctaggagccatgagggtgatggtggggacaggaggaagctcagatgtaagtcgatcccccaacatggtttgcagggagccccttctttgggtgataaagccagcacattagccccgcttgcctgcgcggtctgtgtttgcacgctattggccggcaccagaaggagaggggggtactggcgccaaaccgctgaccacccaaacccatgagccctgtgtggcctcacctcccactgggtctcctccagcgcggggccgaagctggtcttctccctctcgtaggacttgagcttgttgccgcctttgggctccgggccctccagctcgtccctgcagcgccgcggccgctcctcgtaggccaggctggaggcaagctccttctcctcaaagctgcgctgcagcttctggagggcgccctccctctccaacagcttctgctccagctcctggatgctgcactcgtccgtggagatgggggagcggacacacgaggggcccttgtctgccttgttcgagtggcccagcttgctacctccgtcggagaaggacagagccttcaggctcatcatgttgctgtcctggaggacgatgccctgggtgatgttgtgggcggagcccccaaaacggcttgtgggtcccacgggtgtgaccagcgggtccagctggtagctgctgctggtgctgtgtgtgggcaggctggacatggagttccggccggagtctgacagcgccccagagcacaggccaggcttcagctcctgctccttgggcttgtctggaggggcggggtgcagctggtggctggcactctccggggaggagtgcaggatggctcctgaccgtggcagcacaggcttgaaggctgtgggcctcactgcacccttctcggagccctgtagaggaaaaggaccgcggtgactcatgcctcccctgcgcgcgcattgcaccctccctccccaggcacgcgtgccgaccttgagccagtctgggctctctgagcgcacgcagcacccctcttggtcaattgtctcagcagaccttgcctgttgctttgaagcagctgaatgtcatctctcttaggaaggaaaaaccctaatggcgacttgggcactttgttctatgaaatagcaacctgccaccagcttgccccagccctcccgaggtgataaataccatcttgaggctcctgctctaggtctctgtgtggggcaagttagggcatcaggctggccgagcttgctgtccctctttaggtccatcccttcttcctgttcactacctcttccattaagcctggagcaaggacacggacctggcctccttacagggttgggaggctcactccaaatcacgatccttttttaaaactgtaattttccctggtagagtgcttcacagtttacaagccccttaatttgaaaagctgaatgctctatgcaaacataagaggctctttcctagtaaatcaaagccggggattcatttccccagggcaaggggcagaggactgaataggaaaattgatttcagtgtccactcgtgggacgcacgagggcttgagctggtgtgagggctggatttctcagtgcctgggcctcctttgccctaatctctggtaaatggatgacaaaactccagcctgtattcaaagatgcccccaggcgcagcttgaacaaggagctaatgcacaccagggcagcaaatgagaagaccgcacccacccccacgagtctcccggggagagaagcggttaactcccggcctgcatcctcttcatctgtgcttccagatgagaacagggctccctctccttcccgaggcttggcaaacgcctggatcctacgttgacaatccagctacatttcagtgggactccagaaagctcacatatcccctgtgctctttgcttatggcctgacccaagacttctgcttcagggggactgagcgatgctctaattcctttgtgaaacgtttgatctctgcggtgtggccacaggcttccgccggcacccctgccgctctggttttgaggagtctgaatgctcaggtcaccactccccctgaacccccaggctctccacccccattttgctttctcctgcgtttccaacccacttacccttgccagcgacccccgcttaccatttctagctgattggagaagggcatgagcttggggggtgtggacgggtcaaagtccaccccagcctggccccctaaatccccgctggacagtgccgtgtaatctgggtgatgggagccccgggctttctggctgaccttgatgtagaagaagtcttcgctcttgcccattttggagctggacttgccgtgaccggagtcctgggagaagccaaacctcagcagcccgtcggaataccggttgagcttcttgaggtgggaggacttgcgcagcttgtactgcgaagcccggcagtgcttgctgtggaagctgtggccggagatgaggctactgacgctgcccatggtgactcggggctgaggatggggcagggccgggcagggtcttggaaaggctgtggcagcaaggggcagtcgtggctccgtgaggggactgaggtcatagcaaagccctcacagagcctgcgagagccgtagacctggaagaagacacaagacagaagtcagcgtgggtgggagtggtcacagcacagtgctgattccgacttccagatctgggcagtagagaaacacatgtcagagcccagcactgtctccaccaccgggtgctccgttccttcttcctttgtctgataattcaacaaacattcactgggagcccacttcatgcaagcctctactctaaacactcgggacccagcaagtgactaaaacagtcacagtccctgcattcctggaacttagggttacagtggtgtggagaggccagaagcaaaccaatgtatactatagcaagtggtaccagtgctgcgggtaccagtgctgcgggtaccagtgctgcgggggaaactggagcagagaggacagaacagaattctgggaggtggctgttttatgcaggaaggtcttcttttgttagggtagcattttaaaaggcttcaagaaaatgagggggcagccaggcatgatggttcacacctgtaatcccagcactctgggaggctgaggtgggcagattgcttgagtccaggagttcgagaccagcctgtgcaacttagagaaaccccatttctactaaaaatacaaaaattagccgggcgtagtggtgcacacctgtaatcccagctacttgggagggtgaggtgggagaatcgtctgagccccggggatcaaggctgcagtgagccacaattgtgccactgcactccagcctgggcctgactcaaaaaaaaaaaaaaaaaa

SEQ ID NO: 95 - DNA, 19 bp, Human
caggctctgtgagggcttt

SEQ ID NO: 96 - DNA, 19 bp, Human
cgctgacttctgtcttgtg

SEQ ID NO: 97 - DNA, 19 bp, Human
cccggtggtggagacagtg

SEQ ID NO: 98 - DNA, 19 bp, Human
ccacctcaccctcccaagt

SEQ ID NO: 99 - DNA, 19 bp, HUMAN
tcagcctcccagagtgctg

SEQ ID NO: 100 - DNA, 19 bp, Human
gctccagtttcccccgcag

SEQ ID NO: 101 - DNA, 19 bp, Human
cacttgctgggtcccgagt

SEQ ID NO: 102 - DNA, 19 bp, Human
gagacagtgctgggctctg

SEQ ID NO: 103 - DNA, 3629 bp, Human
agaaatgtaaatgtggagccaaacaataacagggctgccgggcctctcagattgcgacggtcctcctcggcctggcgggcaaacccctggtttagcacttctcacttccacgactgacagccttcaattggattttctccatctagcggagccgggggctgcctggaaagatcgctccaggaaggacaaaggtccggaagttgtgggaccttagcagcttgggctccccggatcacccccaaatgatcatttcggaatggagccccagttttcactaggatgccatgggctctaaaatatacagctatgagttctcaatgtttcgagatccaaaagtctcagacctcaatgctttgtgcatcttttatttcaaggattccctacgcccagcaccgggtggatgtgcaaagaagtacgctttaggccggctcaaggttccccaaagctccactcctctgcctaggcgttcaactttgagttcggatggtcctaacatccccatcatctacacccaggtctcccaacaatgcaactcctatgatgatccctctagccaagcttccatcccactcacccccaaactcgctaagtccccactgccccacccccagccccagcgattttcccgagctgaaaatacacggagccgagagcccgtgactcagagaggactcatcaagttcagtcaggagcttacccaatccagggaagcgtgtcaccgtcgtggaaagcacgctcccagcccgaacgcaaagtgtccccggagcccagcagctacctgctccctggacggtggctctagacttttgagaagctcaaaacttttagcgccagtcttgagcacatgggaggggaaaaccccaatcccatcaacccctgcgaggctcctggcacaaagctggacagtcgccatgacaagtaagggcaagtaatccgcctgccggaggaagcaaaggaaatggagttggggaggagggtgcagagtcaggattctcgccgacctggtgccgtagatactaacattttggggtggaaaattctgcaagccagagctgtgagggcagaattggtggaaatcattttggaggaatcctgcattgtgtcaaatatgaagggtggaaggaagaaagcttttgcgtttgctctcagctggatcctttcttctcatcagttaaaatgtcattttttaggaaggctttccgtaatatcacaccctaacgttttctcccagatactttatatcacaccatcttatttaatctccttcacaacccttatcactctgataagatttatttgttcattgctttcagtacatggaaacgtaagccttatgaggatatagaatttttctactatcttattcattgttgtattcctgagtgcctatatcagtgctgggtagcaagtaagagctcgataataaatattttttgaatgagggagacaggtctgaagcctggagaatgagatgcagaagaggtgcaagacctgctgcgccctctgcaggcggcgggggggcggtgcaggtgctttaagaattaccgcgggactcggtagggggagcgtaggcgcttctcgccaagatagaagcgttcagactacaactcccagcagccacgaggagccctagggcttgatgggaacgggaaaccttctaacctttcacgtcccggctccgcgggttccgtgggtcgcccgcgaaatctgatccgggatgcggcggcccaatcggaaggtggaccgaaatcccgcgacagcaagaggcccgtagcgacccgcggtgctaaggaacacagtgctttcaaaagaattggcgtccgctgttcgcctctcctcccgggagtcttctgcctactcccagaagaggagggaagcacatgtgggtttctttagctctgcgtcggatccctgagaacttcgaagccatcctggctgaggctaatctccgctgtgcttcctctgcagtatgaagactttggagactcaaccgttagctccggactgctgtccttcagaccaggacccagctccagcccatccttctccccacgcttccccgatgaataaaaatgcggactctgaactgatgccaccgcctcccgaaaggggggatccgccccggttgtccccagatcctgtggctggctcagctgtgtcccaggagctacgggagggggacccagtttctctctccactcccctggaaacagagtttggttcccctagtgagttgagtcctcgaatcgaggagcaagaactttctgaaaatacaagccttcctgcagaagaagcaaacgggagcctttctgaagaagaagcgaacgggccagagttggggtctggaaaagccatggaagatacctctggggaacccgctgcagaggacgagggagacaccgcttggaactacagcttctcccagctgcctcgatttctcagtggttcctggtcagagttcagcacccaacctgagaacttcttgaaaggctgtaagtgggctcctgacggttcctgcatcttgaccaatagtgctgataacatcttgcgaatttataacctgcccccagagctgtaccatgagggggagcaggtggaatatgcagaaatggtccctgtccttcgaatggtggaaggtgataccatctatgattactgctggtattctctgatgtcctcagcccagccagacacctcctacgtggccagcagcagccgggagaacccgattcatatctgggacgcattcactggagagctccgggcttcctttcgcgcctacaaccacctggatgagctgacggcagcccattcgctctgcttctccccggatggctcccagctcttctgtggcttcaaccggactgtgcgtgttttttccacggcccggcctggccgagactgcgaggtccgagccacatttgcaaaaaagcagggccagagcggcatcatctcctgcatagccttcagcccagcccagcccctctatgcctgtggctcctacggccgctccctgggtctgtatgcctgggatgatggctcccctctcgccttgctgggagggcaccaagggggcatcacccacctctgctttcatcccgatggcaaccgcttcttctcaggagcccgcaaggatgctgagctcctgtgctgggatctccggcagtctggttacccactgtggtccctgggtcgagaggtgaccaccaatcagcgcatctacttcgatctggacccgaccgggcagttcctagtgagtggcagcacgagcggggctgtctctgtgtgggacacggacgggcctggcaatgatgggaagccggagcccgtgttgagttttctgccccagaaggactgcaccaatggcgtgagcctgcaccctagcctgcctctcctggccactgcctccggtcagcgtgtgtttcctgagcccacagagagtggggacgaaggagaggggctgggccttcccttgctctccacgcgccacgtccaccttgaatgtcggcttcagctctggtggtgtgggggggcgccagactccggcatccctgatgatcaccagggcgagaaagggcagggaggaacggagggaggtgtgggtgagctgatataaaaaggtttttatg

SEQ ID NO: 104 - DNA, 19 bp, Human
ggcggattacttgccctta

SEQ ID NO: 105 - DNA, 19 bp, Human
cctccccaactccatttcc

SEQ ID NO: 106 - DNA, 19 bp, Human
cacaatgcaggattcctcc

SEQ ID NO: 107 - DNA, 19 bp, Human
gtgtctccctcgtcctctg

SEQ ID NO: 108 - DNA, 19 bp, Human
ccccctcccgtagctcctg

SEQ ID NO: 109 - DNA, 19 bp, Human
ggcttcagacctgtctccc

SEQ ID NO: 110 - DNA, 19 bp, Human
cctcacagctctggcttgc

SEQ ID NO: 111 - DNA, 19 bp, Human
accctagcctgcctctcct

SEQ ID NO: 112 - DNA, 1359 bp, HUMAN
  misc_feature (521)..(521): n is a, c, g, t or u
aactgtggctgtcgtttgtcccagcgagcggcaagtggggagcgcaagaaaaagtagtaattgttaggagatctcgtctcctaactcggggagttctccaagagagttctccaacttctgtccgaggactggagacgcagagtactcacaagtccggcacttgagtggctgcggctgcgacggcaatttagtgacacgcggctcctttatctcccgactttttctctggcatcaaactcgtgcatgctgtgaagctctcagtccctcgctgagttccactgccccctcactaaaaccctggggctagtcggacctctcggtacagcccattcccaggaagggtcggacttctgctggctccgtactgcgggcgacagtcatctccgaagatctcagatcccagtagtgcgggagcactagccgcctcgggttgtagatttcactcaaatgacaagtgaagctagttctcattgagaatgccacccacacgcacaaatacaacaaggcttaccccgattagngacagntgtggactnnccagacagntttntaacaangcctcctcntctagggnngncccgcccaaagcccctccctaccccaattaccggacctcaaggtctagctgtgctaatgactcatagtttatntcanccatgtataaagaatgcagaagactccagaaggtgggggagccactagaggattccatccaggacaccacatttaattgttatgatgtcttgtgctcctcttggctgtgagagtttctcagattttccttgtatttgatgaccttgacagtccttaggagtactgagatgggggtttcacgatattgcccaggctggtcccaaactcctggcctcaagctatcctcctgccttgacgttcgaaagcactnagattttcctntctgtcctgccgccatgtgaagaaanatgtgtttgcttccccttccnccgtgattgtanttttcctgcagcctccccagccaagctgaactgcaatgaggaagcaataaaggactntgagcaagagaacgatgtatataggccattgttttggaagattcatcncaaattcatgtgcaagtagattggaggaaggaagtatcaacanaaanaaaggccaattatgagaccattgcaatactatatgatgagagcatcagagncaataaccaaggtnttatagataccaagtgaggaatctgggattanaagtcagtntatttcattggaaaggctgttttcagtntttttcactggaaaagttgtcatcctgtgtctttnttatagtacatatattncagtaataaaactttattttccttttcaaaaaaaaaaaaaaaaaaaa

SEQ ID NO: 113 - DNA, 20 bp, Human
gtggtgtcctggatggaatc

SEQ ID NO: 114 - DNA, 20 bp, Human
tcccagattcctcacttggt

(19) United States
(12) Patent Application Publication
(10) Pub. No.: US 2009/0092988 A1
(43) Pub. Date: Apr. 9, 2009

(54) Modulating Gene Expression with agRNA and Gapmers Targeting Antisense Transcripts

(76) Inventors: Jacob C. Schwartz, Dallas, TX (US); Scott T. Younger, Dallas, TX (US); Bethany A. Janowski, Dallas, TX (US); David R. Corey, Dallas, TX (US)

Correspondence Address: RICHARD ARON OSMAN, 4070 CALLE ISABELLA, SAN CLEMENTE, CA, 92672, US

(21) Appl. No.: 12/246,421

(22) Filed: Oct. 6, 2008

Related U.S. Application Data
(60) Provisional application No. 60/977,631, filed on Oct. 4, 2007.
(60) Provisional application No. 61/030,985, filed on Feb. 24, 2008.

(51) Int. Cl. C12Q 1/68

(52) U.S. Cl. 435/6

(57) ABSTRACT

Gene expression is selectively modulated in the genome of a mammalian cell determined to be in need thereof by determining the presence of an encoded antisense transcript overlapping a promoter of the target gene; contacting the transcript with an agRNA or gapmer complementary to a portion of the transcript upstream relative to the transcription start site of the gene; and detecting a resultant modulation of expression of the target gene.

### CROSS-REFERENCE TO RELATED APPLICATIONS

This application claims priority to U.S. Ser. No. 60/977,631, filed Oct. 04, 2007, and to Ser. No. 61/030,985, filed Feb. 24, 2008.

This work was made with Government support under grants awarded by the NIH (NIGMS 60642 and 73042 and NIBIB F31 EB005556-01). The Government has certain rights in this invention.

### FIELD OF THE INVENTION

The field of the invention is modulating gene expression using antigene RNA or gapmers targeting an antisense transcript overlapping a promoter of the gene.

### BACKGROUND OF THE INVENTION

Recent studies have reported that duplex RNAs complementary to promoter regions can repress or activate gene expression^{9,10}. The mechanism of these promoter directed RNAs (pdRNAs) has been obscure. Other recent work using microarray analysis has revealed networks of non-coding transcripts surrounding regions of the genome that code for mRNA¹¹⁻¹⁴. The function of these RNA networks is also not understood. Here we link these two sets of enigmatic results. We identify a network of antisense transcripts at the promoter for progesterone receptor (PR). We show that pdRNAs bind antisense transcripts and that activation of gene expression by pdRNAs requires expression of antisense transcript. pdRNAs recruit argonaute proteins to both the PR promoter and to the PR antisense transcript. pdRNAs shift localization of the multifunctional protein heterogenous ribonucleoprotein-k (hnRNP-k) from chromosomal DNA to the antisense transcript. These data demonstrate that pdRNAs can significantly remodel protein interactions at gene promoters. Our results link the action of pdRNAs with recognition of antisense transcripts and provide a mechanism for RNA-mediated gene regulation at promoters.

We have observed that pdRNAs complementary to target sequences within gene promoters can either selectively activate or inhibit gene expression in mammalian cells. pdRNAs recruit argonaute proteins to promoter DNA and reducing levels of argonaute protein blocks pdRNA activity. Argonaute proteins are known to mediate recognition of mRNA by small RNAs during post-transcriptional RNA^{15,16}, and we hypothesized that their pdRNAs might also have RNA targets. There were, however, no known RNA targets for our pdRNAs. We disclose that pdRNAs are recognizing previously undiscovered transcripts that overlap gene promoters, and that we can use targeted pdRNAs, including antigene RNA and gapmers, to modulate expression of target genes.

### SUMMARY OF THE INVENTION

The invention provides a general method of selectively modulating expression of a target gene in the genome of a mammalian cell determined to be in need thereof, comprising: (a) contacting the transcript with an exogenous gapmer or double-stranded agRNA; and (b) detecting a resultant modulation of expression of the target gene, the gapmer comprising a DNA insert complementary to a portion of the transcript upstream relative to the transcription start site of the gene, and the agRNA being 18-28 bases and complementary to a portion of the transcript upstream relative to the transcription start site of the gene;

In particular embodiments of each aspect and embodiment of the invention, the expression is modulated and/or detected at the level of target gene transcription.

In particular embodiments, the method comprises an antecedent step of determining the presence of an encoded antisense transcript overlapping a promoter of the target gene, which step may be implemented in silico by examining transcriptional data to identity the antisense transcript, and/or in vitro by using 5′-RACE/3′-RACE (Rapid Amplification of Complementary Ends) to experimentally identify the antisense transcript.

In particular embodiments, the DNA insert is complementary to a portion of the transcript more than 100, more than 200, or more than 1,000 bases upstream relative to the transcription start site of the gene.

In particular embodiments, the agRNA, gapmer and/or DNA insert is a priori not known to be a modulator of the target gene, and/or the antisense transcript is a priori not known to overlap the promoter of the target gene.

In particular embodiments, the modulation is methylase-independent, and/or the agRNA or DNA insert is complementary to a portion of the transcript free of CpG islands. In related embodiments, the method further comprises the step of confirming that the modulation is methylase-independent, and/or the step of confirming that the agRNA or DNA insert is complementary to a portion of the transcript free of CpG islands.

In particular embodiments the contacting step is free of viral transduction.

In particular embodiments the contacting step is implemented by contacting the cell with a composition consisting essentially of the agRNA or DNA insert, and/or a composition comprising the agRNA or DNA insert at 1-100 nanomolar concentration.

In particular embodiments, the detecting step is implemented by detecting at least a 25%, preferably at least a 50%, more preferably at least a 200% increased expression of the target gene, or at least a 50%, preferably at least a 75%, more preferably at least a 90% decreased expression of the target gene, relative to a negative control.

In particular embodiments, no more than one portion of the antisense transcript is targeted.

Additional embodiments encompass combinations of the foregoing particular embodiments, and methods of doing business comprising promoting, marketing, selling and/or licensing a subject embodiment.

### DETAILED DESCRIPTION OF SPECIFIC EMBODIMENTS OF THE INVENTION

In one specific embodiment, the invention provides a general method of selectively modulating transcription of a target gene in the genome of a mammalian cell determined to be in need thereof, comprising: (a) determining the presence in the genome of an encoded antisense transcript overlapping a promoter of the target gene; (b) contacting the transcript with an exogenous, double-stranded agRNA of 18-28 bases and complementary to a portion of the transcript upstream relative to the transcription start site of the gene; and (c) detecting a resultant modulation of transcription of the target gene.

In another specific embodiment, the invention provides a general method of selectively modulating expression of a target gene in the genome of a mammalian cell determined to be in need thereof, comprising: (a) contacting the transcript with an exogenous gapmer comprising a DNA insert complementary to a portion of the transcript upstream relative to the transcription start site of the gene; and (b) detecting a resultant modulation of expression of the target gene.

The recited mammalian cell is preferably human, and may be in vitro (e.g. a cultured cell), or in situ in a host. Examples of cultured cells include primary cells, cancer cells (e.g. from cell lines), adult or embryonic stem cells, neural cells, fibroblasts, myocytes, etc. Cultured human cells commonly used to test putative therapeutics for human diseases or disorders can be used to screen agRNAs or gapmers that target antisense transcripts for therapeutic affect (e.g. induction of apoptosis, cessation of proliferation in cancer cells, etc.). When the cell is in situ, the host may be any mammal, such as a human, or an animal model used in the study of human diseases or disorders (e.g. rodent, canine, porcine, etc. animal models).

The mammalian cell may be determined to be in need of modulated expression of the target gene using routine methods. For example, reduced levels of a target gene expression and/or protein relative to desired levels may be directly measured. Alternatively, the need for increased or decreased expression may be inferred from a phenotype associated with reduced or increased levels of a target gene product.

The recited determining step may be implemented in silico, for example, by examining transcriptional data to identity the antisense transcript, and/or in vitro or in vivo, for example, by using 5′-RACE/3′-RACE to experimentally identify the antisense transcript.

In one embodiment, the determining step for targeting new genes may be implemented by steps:

1: Examining one or more transcriptional databases such as the FANTOM or ENCODE databases to derive a map of transcripts (the transcriptional landscape) overlapping a promoter of the mRNA of the target gene; and/or

2. Using 5′-RACE/3′-RACE to experimentally characterize the transcriptional landscape overlapping the promoter;

3. Designing multiple (e.g. 3-10) duplex RNAs that overlap both the promoter region and one or more antisense transcripts (noncoding transcripts being preferred);

4: For identified active agRNAs, using biotin-labeled agRNAs to confirm the antisense RNA being bound by the agRNAs.

The recited agRNAs optionally have 3′ di- or trinucleotide overhangs on each strand. Methods for preparing dsRNA and delivering them to cells are well-known in the art (see e.g. Elbashir et al, 2001; WO/017164 to Tuschl et al; and U.S. Pat. No. 6,506,559 to Fire et al). Custom-made dsRNAs are also commercially available (e.g. Ambion Inc., Austin, Tex.). The dsRNA may be chemically modified to enhance a desired property of the molecule. A broad spectrum of chemical modifications can be made to duplex RNA, without negatively impacting the ability of the agRNA to selectively modulate transcription. In one embodiment, the agRNA comprises one or more nucleotides having a 2′ modification, and may be entirely 2′-substituted. A variety of 2′ modifications are known in the art (see e.g. U.S. Pat No. 5,859,221; U.S. Pat No. 6,673,611; and Czauderna et al, 2003, Nucleic Acids Res. 31:2705-16). A preferred chemical modification enhances serum stability and increases the half-life of dsRNA when administered in vivo. Examples of serum stability-enhancing chemical modifications include phosphorothioate internucleotide linkages, 2′-O-methyl ribonucleotides, 2′-deoxy-2′-fluoro ribonucleotides, 2′-deoxy ribonucleotides, “universal base” nucleotides, 5-C-methyl nucleotides, and inverted deoxyabasic residue incorporation (see e.g. US Pat Pub No. 20050032733). The agRNA may optionally contain locked nucleic acids (LNAs) to improve stability and increase nuclease resistance (see e.g. Elmen et al, 2005 Nucleic Acids Res. 33:439-47; and Braasch et al, 2003 Biochemistry. 42:7967-75). Another type of modification is to attach a fluorescent molecule to the agRNA, for example, TAMRA, FAM, Texas Red, etc., to enable the agRNA to be tracked upon delivery to a host or to facilitate transfection efficiency determinations.

The gapmers are designed to target various regions of the antisense transcript emphasizing those sequences closest to the transcription start site of the sense gene. We biased selection toward sequences with a melting temperature around 60° C., a GC content between about 25% and 75%, and about 20 nucleotides long because historically, oligonucleotides with these properties are easier to work with. The 5 nucleotides at the 5′ and 3′ ends should be modified nucleotides such as 2′ MOE or 2′OMe or Locked Nucleic Acid bases (LNA). The outside modified nucleotides of the gapmer provide protection from nucleases, and the central DNA region hybridizes to corresponding RNA sequences in the cell. The subsequent DNA-RNA hybrid is recognized by the nuclease RNase H, thereby destroying the RNA molecule.

The agRNA or DNA insert of the gapmer may be complementary to any portion of the transcript upstream from the promoter of the target gene, insuring that the binding target of the DNA insert is the antisense transcript, and not a transcript of the target gene. In particular embodiments, the agRNA or DNA insert is complementary to a portion of the transcript more than 100, more than 200, or more than 1,000 bases upstream relative to the transcription start site of the gene.

While multiple portions of the target promoter can be targeted, highly efficient increased synthesis of the target transcript can be achieved by targeting just a single region of the target promoter. In particular embodiments, no more than one portion of the transcript is targeted.

In particular embodiments, such as screening assays for agRNAs or gapmers, the agRNA or gapmer is a priori not known to be a modulator of the target gene. In particular embodiments, such as identifying novel gene targets of the method, the antisense transcript is a priori not known to overlap the promoter of the target gene.

In particular embodiments, the modulation is methylase-independent, wherein synthesis of the target transcript is modulated independently of, and without requiring effective methylation. In particular embodiments, the agRNA or DNA insert of the gapmer is complementary to a portion of the antisense transcript outside of (not contained within) a CpG island. Algorithms for identifying CpG islands in genomic sequences are known (e.g. see Takai and Jones, 2002 Proc Natl Acad Sci USA. 99:3740-5; and Takai and Jones 2003 In Silico Biol. 3:235-40). In another embodiment, the target portion does not include a CG dinucleotide. In related embodiments, the method further comprises the step of confirming that the modulation is methylase-independent, and/or the step of confirming that the DNA insert of the agRNA or gapmer is complementary to a portion of the transcript outside a CpG island.

In certain embodiments, the target gene is known to encode and/or express one or more isoforms, and the method selectively modulates, including increases or decreases, the relative expression of the isoforms, which may be in reciprocal coordination, e.g. one increases, while the other decreases. The isoforms may share the same promoter and/or transcription start site, or they may have different promoters and/or transcription start sites. Accordingly, in various embodiments, the recited promoter is (1) the promoter of a target gene first transcript, (2) the promoter of an isoform of the target gene first transcript, or (3) is the promoter of both the target gene first transcript and of an isoform thereof.

For example, the methods can be used to increase expression of a first target gene transcript by directing agRNAs or gapmers to an antisense transcript overlapping the transcription start site of an isoform thereof. For example, where synthesis of the first transcript is increased, and synthesis of the isoform is inhibited, the method effectively and selectively modulates relative isoform synthesis in the host cell. Hence, increased synthesis of predetermined desirous or underexpressed isoforms can be coupled with decreased synthesis of predetermined undesirable or overexpressed isoforms. This embodiment can be used to effect a predetermined isoform switch in the host cells.

Significant modulation of target gene expression may be achieved using nanomolar (submicromolar)or picomolar (subnamomolar) concentrations of the agRNA or gapmer, and it is typically preferred to use the lowest concentration possible to achieve the desired resultant increased synthesis, e.g. agRNA or gapmer concentrations in the 1-100 nM range are preferred; more preferably, the concentration is in the 1-50 nM, 1-25nM, 1-10 nM, or picomolar range. In particular embodiments, the contacting step is implemented by contacting the cell with a composition consisting essentially of the agRNA or gapmer.

A variety of methods may be used to deliver the agRNA or gapmer inside the cell. For cells in vitro, delivery can often be accomplished by direct injection into cells, and delivery can often be enhanced using hydrophobic or cationic carriers such as Lipofectamine™ (Invitrogen, Carlsbad, Calif.). Alternatively, the cells can be permeabilized with a permeabilization agent such as lysolecithin, and then contacted with the agRNA or gapmer.

For cells in situ, cationic lipids (see e.g. Hassani et al, 2004 J Gene Med. 7:198-207) and polymers such as polyethylenimine (see e.g. Urban-Klein, 2005 Gene Ther. 12:461-6) have been used to facilitate agRNA an dgapmer delivery. Compositions consisting essentially of the agRNA or gapmer (in a carrier solution) can be directly injected into the host (see e.g. Tyler et al, 1999 PNAS 96:7053-7058; McMahon et al, 2002 Life Sci. Jun. 7, 2002;71(3):325-37.). In vivo applications of duplex RNAs are reviewed in Paroo and Corey, 2004 Trends Biotechnol 22:390-4.

Viral transduction can also be used to deliver agRNAs to cells (e.g. lentiviral transduction). However, in certain embodiments, it is preferred that the contacting step is free of viral transduction and/or that the agRNA is not attached to a nuclear localization peptide.

The detecting step is implemented by detecting a significant change in the expression of the target gene, preferably by detecting at least a 10%, 25%, 50%, 200% or 500% increased expression of the target gene, or at least a 10%, 25%, 50%, 75%, or 90% decreased expression of the target gene, relative to a negative control, such as basal expression levels.

Detection may be effected by a variety of routine methods, such as directly measuring a change in the level of the target gene mRNA transcript, or indirectly detecting increased or decreased levels of the corresponding encoded protein compared to a negative control. Alternatively, resultant selective modulation of target gene expression may be inferred from phenotypic changes that are indicative of increased or decreased expression of the target gene.

As disclosed and exemplified herein, by exploiting a hitherto unappreciated endogenous mechanism for selective regulation of gene expression, our methods are generally applicable across a wide variety of target genes, promoter regions, agRNAs, gapmers, mammalian cell types and delivery conditions. While conditions whereby a given agRNA or gapmer selectively modulates expression of a given target gene may be confirmed empirically (e.g. pursuant to the protocols described herein), we have consistently found modulating, including activating and inhibiting, agRNAs and gapmers for every mammalian gene we have studied; and our data indicate that mammalian cells are generally amenable to target gene selective modulation of target gene expression using these methods.

Additional embodiments encompass combinations of the foregoing particular embodiments, and methods of doing business comprising promoting, marketing, selling and/or licensing a subject embodiment.

agRNA Examples

Specific gene targets and dsRNA sequences that selectively increase transcript synthesis are listed in Table 1. Only one strand (shown 5′ to 3′) of each dsRNA is shown. Additionally the dsRNAs had 3′-dithymidine overhangs on each strand.

Our studies establish a novel technique for isolating endogenous targets of small RNAs and demonstrate that the many promoters contain an overlapping antisense ncRNA transcript which serves as a substrate for agRNAs; that agRNAs interact directly with the antisense transcript and not with chromosomal DNA; that agRNAs recruit Argonaute to the antisense transcript; and that antisense transcripts are targets for agRNAs.

By way of example, an antisense RNA transcript was identified in the promoter of progesterone receptor (PR) gene: (a) The transcription start site of PR mRNA was determined by 5′ RACE. (b) Quantitative RT-PCR primers were designed targeting every exon boundary in the PR transcript and walking across the PRB transcription start site and into the promoter. (c) No reverse transcriptase controls ensure that detected product is RNA and not contaminating DNA. (d) RNA transcript was detected in the PR promoter ranging from 10 to 1000 fold lower expression than the main PR transcript in polyA RNA purified from T47D cells. (e) RNA transcript at similar levels was detected in polyA RNA purified from MCF7 cells. (f) Multiple noncoding antisense transcription start sites were identified by 5′ RACE. (g) Targetable ncRNAs are spliced and map to as far 70 kb upstream of the PRB transcription start site. (h) Expression levels of each antisense transcript relative to PR mRNA were analyzed by qPCR.

agRNAs were shown to bind directly to the antisense transcript: a) Biotinylated agRNAs inhibit gene expression. (b) Biotinylated agRNAs activate gene expression. (c) Sense strand of inhibitory agRNA binds directly to the antisense transcript. (d) Sense strand of activating agRNA binds directly to the antisense transcript.

agRNAs were shown to recruit Argonaute to the antisense transcript: (a) RNA immunoprecipitation shows inhibitory agRNA recruits Argonaute to the antisense transcript. (b) RNA immunoprecipitation shows activating agRNA recruits Argonaute to the antisense transcript.

Below are exemplary agRNAs targeting antisense transcripts—bold nucleotides mark the transcription start site (+1).

I. Tumor Suppressor Candidate Protein 4 (NM_{—}006545) Promoter (starting at −200)

(SEQ ID NO:01)

CCAGTCGACGGCCGGCGGCCTGTCAACGTGTACCCATGTCT
GAACTGGTACCAATCGCTGGCCCGCCTTCCAGGTAGGAGGC
GCAAAGCCATGTAAGACTACAAATCCCAGCGTGTACCACGC
CGTCGCCGGCAGTAGAGCCAGCTGGGAGGGCGCGGAGCACT
ATGGAAATTGTAGTTCCCTGCTGCGGTCCCAGTTACAGCGT
GAATCCCTTAGCGCACCGCCTCCCCAAGTGCTGCCAGCATG
CTGCC +50

| | |
|---|---|
| agRNAs | |
| TUSC-13 | 5′ GCGGUCCCAGUUACAGCGUTT 3′ |
| | (SEQ ID NP:02) |
| | 3′ TTCGCCAGGGUCAAUGUCGCA 5′ |
| | (SEQ ID NO:03) |
| TUSC-50 | 5′ GGGCGCGGAGCACUAUGGATT 3′ |
| | (SEQ ID NO:04) |
| | 3′ TTCCCGCGCCUCGUGAUACCU 5′ |
| | (SEQ ID NO:05) |
| TUSC-190 | 5′ GCCGGCGGCCUGUCAACGUTT 3′ |
| | (SEQ ID NO:06) |
| | 3′ TTCGGCCGCCGGACAGUUGCA 5′ |
| | (SEQ ID NO:07) |
| TUSC-150 | 5′ CCAAUCGCUGGCCCGCCUUTT 3′ |
| | (SEQ ID NO:08) |
| | 3′ TTGGUUAGCGACCGGGCGGAA 5′ |
| | (SEQ ID NO:09) |
| TUSC-140 | 5′ CAGGTAGGAGGCGCAAAGCTT 3′ |
| | (SEQ ID NO:10) |
| | 3′ TTGUCCAUCCUCCGCGUUUCG 5′ |
| | (SEQ ID NO:11) |
| TUSC-500 | 5′ CCAGGCCUGGCAAGCACAGTT 3′ |
| | (SEQ ID NO:12) |
| | 3′ TTGGUCCGGACCGUUCGUGUC 5′ |
| | (SEQ ID NO:13) |
| TUSC-380 | 5′ AUCGUCAGCCGUGCUAGUGTT 3′ |
| | (SEQ ID NO:14) |
| | 3′ TTUAGCAGUCGGCACGAUCAC 5′ |
| | (SEQ ID NO:15) |
| TUSC-230 | 5′ AAGACCAGCACCAGGAAUGTT 3′ |
| | (SEQ ID NO:16) |
| | 3′ TTUUCUGGUCGUGGUCCUUAC 5′ |
| | (SEQ ID NO:17) |
| TUSC-977 | 5′ GCACCGGGUGCCAGGAGAATT 3′ |
| | (SEQ ID NO:18) |
| | 3′ TTCGUGGCCCACGGUCCUCUU 5′ |
| | (SEQ ID NO:19) |
| TUSC-973 | 5′ CGGGUGCCAGGAGAACAGGTT 3′ |
| | (SEQ ID NO:20) |
| | 3′ TTGCCCACGGUCCUCUUGUCC 5′ |
| | (SEQ ID NO:21) |

II. Leucine Zipper putative tumor suppressor 1 (NM_{—}021020) Promoter (starting at −200)

(SEQ ID NO:22)

cccagtgaatgtttgttgaatTATCAGACAAAGGAAGAAGGAACG
GAGCACCCGGTGGTGGAGACAGTGCTGGGCTCTGACATGTGTTTC
TCTACTGCCCAGATCTGGAAGTCGGAATCAGCACTGTGCTGTGAC
CACTCCCACCCACGCTGACTTCTGTCTTGTGTCTTCTTCCAGGTC
TACGGCTCTCGCAGGCTCTGTGAGGGCTTTGCTATGACCTCAGTC
CCCTCACGGAGCCACGACTGCCCCTT +50

| | |
|---|---|
| agRNAs | |
| LZTS1-9 | 5′ CAGGCUCUGUGAGGGCUUUTT 3′ |
| | (SEQ ID NO:23) |
| | 3′ TTGUCCGAGACACUCCCGAAA 5′ |
| | (SEQ ID NO:24) |
| LZTS1-52 | 5′ CGCUGACUUCUGUCUUGUGTT 3′ |
| | (SEQ ID NO:25) |
| | 3′ TTGCGACUGAAGACAGAACAC 5′ |
| | (SEQ ID NO:26) |
| LZTS1-150 | 5′ CCCGGUGGUGGAGACAGUGTT 3′ |
| | (SEQ ID NO:27) |
| | 3′ TTGGGCCACCACCUCUGUCAC 5′ |
| | (SEQ ID NO:28) |
| LZTS1-690 | 5′ CCACCUCACCCUCCCAAGUTT 3′ |
| | (SEQ ID NO:29) |
| | 3′ TTGGUGGAGUGGGAGGGUUCA 5′ |
| | (SEQ ID NO:30) |
| LZTS1-550 | 5′ UCAGCCUCCCAGAGUGCUGTT 3′ |
| | (SEQ ID NO:31) |
| | 3′ TTAGUCGGAGGGUCUCACGAC 5′ |
| | (SEQ ID NO:32) |
| LZTS1-400 | 5′ GCUCCAGUUUCCCCCGCAGTT 3′ |
| | (SEQ ID NO:33) |
| | 3′ TTCGAGGUCAAAGGGGGCGUC 5′ |
| | (SEQ ID NO:34) |
| LZTS1-250 | 5′ CACUUGCUGGGUCCCGAGUTT 3′ |
| | (SEQ ID NO:35) |
| | 3′ TTGUGAACGACCCAGGGCUCA 5′ |
| | (SEQ ID NO:36) |
| LZYS1-140 | 5′ GAGACAGUGCUGGGCUCUGTT 3′ |
| | (SEQ ID NO:37) |
| | 3′ TTCUCUGUCACGACCCGAGAC 5′ |
| | (SEQ ID NO:38) |

III. Tumor protein p53 (NM_{—}000546) Promoter (starting at −200)

(SEQ ID NO:39)

TCCACCCTTCATATTTGACACAATGCAGGATTCCTCCAAAATGAT
TTCCACCAATTCTGCCCTCACAGCTCTGGCTTGCAGAATTTTCCA
CCCCAAAATGTTAGTATCTACGGCACCAGGTCGGCGAGAATCCTG
ACTCTGCACCCTCCTCCCCAACTCCATTTCCTTTGCTTCCTCCGG
CAGGCGGATTACTTGCCCTTACTTGTCATGGCGACTGTCCAGCTT
TGTGCCAGGAGCCTCGCAGGGGTTGA +50

| | |
|---|---|
| agRNAs | |
| TP53-18 | 5′ GGCGGAUUACUUGCCCUUATT 3′ |
| | (SEQ ID NO:40) |
| | 3′ TTCCGCCUAAUGAACGGGAAU 5′ |
| | (SEQ ID NO:41) |
| TP53-50 | 5′ CCTCCCCAACTCCATTTCCTT 3′ |
| | (SEQ ID NO:42) |
| | 3′ TTGGAGGGGTTGAGGTAAAGG 5′ |
| | (SEQ ID NO:43) |
| TP53-182 | 5′ CACAAUGCAGGAUUCCUCCTT 3′ |
| | (SEQ ID NO:44) |
| | 3′ TTGUGUUACGUCCUAAGGAGG 5′ |
| | (SEQ ID NO:45) |
| TP53-1480 | 5′ GUGUCUCCCUCGUCCUCUGTT 3′ |
| | (SEQ ID NO:46) |
| | 3′ TTCACAGAGGGAGCAGGAGAC 5′ |
| | (SEQ ID NO:47) |
| TP53-1260 | 5′ CCCCCUCCCGUAGCUCCUGTT 3′ |
| | (SEQ ID NO:48) |
| | 3′ TTGGGGGAGGGCAUCGAGGAC 5′ |
| | (SEQ ID NO:49) |
| TP53-540 | 5′ GGCUUCAGACCUGUCUCCCTT 3′ |
| | (SEQ ID NO:50) |
| | 3′ TTCCGAAGUCUGGACAGAGGG 5′ |
| | (SEQ ID NO:51) |
| TP53-140 | 5′ CCUCACAGCUCUGGCUUGCTT 3′ |
| | (SEQ ID NO:52) |
| | 3′ TTGGAGUGUCGAGACCGAACG 5′ |
| | (SEQ ID NO:53) |
| TP53-15649 | 5′ ACCCUAGCCUGCCUCUCCUTT 3′ |
| | (SEQ ID NO:54) |
| | 3′ TTUGGGAUCGGACGGAGAGGA 5′ |
| | (SEQ ID NO:55) |

IV. Nerve Growth Factor Receptor (NM_{—}002507) Promoter (starting at −200)

(SEQ ID NO:56)

GAGAGGCTCTAAGGGACAAGGCAGGGAGAAGCGCAGCGGGG
TGCGGGGAACCGCACGCCCTCCCTTTGCCTCTGCTTCCCAC
CCCGAGGCGGCAgggcgggcgggcgcggttccgggggtggg
cgggctgggcggggcggaggcggggccgcAGCACTGGCTTC
ACCCAGCCTCTCCCGCCCGCAGCCAGAGCGAGCCGAGCCGC
GGCCAGCTCCGGCGGGCAGGGGGGGCGCTGGAGCGCAGCGC
AGCGC

| | |
|---|---|
| agRNAs | |
| NGFR-9 | 5′ GCGAGCCGAGCCGCGGCCATT 3′ |
| | (SEQ ID NO:57) |
| | 3′ TTCGCUCGGCUCGGCGCCGGU 5′ |
| | (SEQ ID NO:58) |
| NGFR-39 | 5′ UUCACCCAGCCUCUCCCGCTT 3′ |
| | (SEQ ID NO:59) |
| | 3′ TTAAGUGGGUCGGUGUGGGCG 5′ |
| | (SEQ ID NO:60) |
| NGFR-200 | 5′ GAGAGGCTCTAAGGGACAATT 3′ |
| | (SEQ ID NO:61) |
| | 3′ TTCUCUCCGAGAUUCCCUGUU 5′ |
| | (SEQ ID NO:62) |
| NGFR-995 | 5′ CCCUGCCUGCAGAGCUCAUTT 5′ |
| | (SEQ ID NO:63) |
| | 3′ TTGGGACGGACGTCTCGAGUA 5′ |
| | (SEQ ID NO:64) |
| NGFR-700 | 5′ GUGGGCACACGUAAGUGCATT 3′ |
| | (SEQ ID NO:65) |
| | 3′ TTCACCCGUGUGCAUUCACGU 5′ |
| | (SEQ ID NO:66) |
| NGFR-520 | 5′ CCUAGGCCUCUGCCCAGGGTT 3′ |
| | (SEQ ID NO:67) |
| | 3′ TTGGAUCCGGAGACGGGUCCC 5′ |
| | (SEQ ID NO:68) |
| NGFR-280 | 5′ CCCUGGUCCCCGGGCCCACTT 3′ |
| | (SEQ ID NO:69) |
| | 3′ TTGGGACCAGGGGCCCGGGUG 5′ |
| | (SEQ ID NO:70) |
| NGFR-160 | 5′ GUGCGGGAACCGCACGCCTT 3′ |
| | (SEQ ID NO:71) |
| | 3′ TTCACGCCCCUUGGCGUGCGG 5′ |
| | (SEQ ID NO:72) |

### Gapmer Examples

We chose to initially characterize the mechanism of pdRNA action using PR as a model gene because we have discovered pdRNAs that activate or inhibit its expression in different cellular contexts. pdRNAs complementary to target sequences within the PR gene promoter inhibit transcription of PR in T47D breast cancer cells^{3,6} a cell line that expresses high levels of PR. Similar pdRNAs activate PR expression in MCF7 breast cancer cells that express low levels of PR¹⁰.

We used 5′-RACE to search for undiscovered sense transcripts that initiate upstream from the transcription start site of the PR gene. 5′-RACE is a PCR-based method for cloning the 5′ end of mRNA transcripts. We used a version of 5′-RACE that selects for full length RNA with the 5′ cap intact¹⁷. To maximize detection of transcripts we used multiple primer sets to amplify regions, both upstream and downstream of the previously determined transcription start sited^{18,9}. Although we sequenced 60 clones for T47D cells and 62 clones for MCF7 cells, we did not identify transcripts initiating upstream of the previously determined^{18,19} transcription start site.

To expand our search for transcripts at the PR promoter, we employed RT-PCR using primers designed to detect transcripts that overlapped the promoter. These primers could detect transcripts that were either sense or antisense relative to PR mRNA. This experiment detected RNA overlapping the PR promoter. Quantitative PCR (qPCR) with multiple primer pairs complementary to the PR promoter revealed that RNA levels at the PR promoter in either MCF7 or T47D cells were 10-1000 fold below PR mRNA levels.

Our detection of RNA at the PR promoter, combined with our inability to detect sense transcripts, suggested that transcription might be occurring in the antisense direction. To test this hypothesis we performed 5′-RACE using primers complementary to potential antisense transcripts. Sequencing the 5′-RACE products revealed the existence of four antisense RNA transcripts at the PR promoter, two of which were similar. Antisense transcripts AT1, AT2-T47D (found only in T47D cells), and AT2-MCF7 (found only in MCF7 cells) overlapped the region targeted by pdRNAs, making them targets for direct physical interactions with pdRNAs.

The closely related AT2-T47D and AT2-MCF7 antisense transcripts were the most highly expressed and were chosen for further study. We subsequently used PCR-based cloning with multiple primer sets to identify the full-length sequence of antisense transcripts AT2-T47D and AT2-MCF7. We observed that they are spliced, polyadenylated, and are transcribed over a 70 kB region of genomic DNA. AT2T47D and AT2-MCF7 initiate at different locations 202 bases apart but are otherwise identical.

To assess involvement of antisense transcripts in the regulation of gene expression by pdRNAs, we obtained single-stranded oligonucleotides complementary to sequences shared by antisense transcripts AT2-MCF7 and AT2-T47D. These single-stranded oligonucleotides are “gapmers” containing a central DNA portion designed to recruit RNAse H to cleave their RNA target and flanking 2′-methoxyethyl RNA regions to enhance affinity to target sequences^{20,21}. Gapmers are effective gene silencing agents and are showing substantial promise in Phase II clinical trials²¹. The goal for these experiments was to use gapmers to test the effect of reducing antisense transcript levels on the activity of pdRNAs.

We tested ten gapmers (G1-G10, Table 1) for their ability to reduce levels of antisense transcript AT2 in both MCF7 and T47D cells. Gapmers G1-G3 were complementary to AT2, G4-G10 were not. We identified one gapmer, G1, capable of reducing levels of AT2 in both MCF7 and T47D cells. We also identified a less active gapmer, G2, capable of reducing transcript levels in MCF7 cells.

TABLE 1
Single-stranded oligonucleotide “gapmers”

| | | | |
|---|---|---|---|
| | | | Position |
| Name | Sequence | Complementarity | from TSS |

| | | | | |
|---|---|---|---|---|
| G1 | TGTTAGAAAGCTGTCTGGCC | (SEQ ID NO:73) | Complementary | −20 |
| G2 | GAGGAGGCGTTGTTAGAAAG | (SEQ ID NO:74) | Complementary | −32 |
| G3 | TAGAGGAGGAGGCGTTGTTA | (SEQ ID NO:75) | Complementary | −35 |
| G4 | ACCGGTAATTGGGGTAGGGA | (SEQ ID NO:76) | Noncomplementary | −77 |
| G5 | TGCCAACTCCAGAGTTTCAG | (SEQ ID NO:77) | Noncomplementary | −102 |
| G6 | GGCCAGACAGCTTTCTAACA | (SEQ ID NO:78) | Noncomplementary | −20 |
| G7 | CTTTCTAACAACGCCTCCTC | (SEQ ID NO:79) | Noncomplementary | −32 |
| G8 | TAACAACGCCTCCTCCTCTA | (SEQ ID NO:80) | Noncomplementary | −35 |
| G9 | TCCCTACCCCAATTACCGGT | (SEQ ID NO:81) | Noncomplementary | −77 |
| G10 | CTGAAACTCTGGAGTTGGCA | (SEQ ID NO:82) | Noncomplementary | −102 |

The five 5′ and 3′ nucleotides of each sequence are 2′ methoxyethyl RNA nucleotides. The middle section is DNA. Complementarity refers to whether the sequence is complementary to transcript AT2. The position from TSS refers to the location of the target sequence with respect to the transcription start site for progesterone receptor. Gapmers G6 through G10 are the reverse.

Addition of gapmer G1 to MCF7 cells prevented gene activation by activating pdRNA PR11 (targeted to the −11/+8 sequence at the PR promoter)¹⁰. This result indicates that the antisense transcript is involved in RNA-mediated gene activation. Addition of the less active gapmer G2 or gapmer G7 that was in the sense orientation (i.e. possessed the same sequence as the antisense transcript) did not prevent activation of PR expression. Addition of gapmer G1 to T47D cells did not significantly affect gene silencing by inhibitory pdRNA PR9 (targeted to the −9/+10 sequence at the PR promoter) 6. The inability of gapmer G1 to reverse gene silencing is consistent with the antisense transcript being 4.5 fold more prevalent in T47D cells than in MCF-7 cells, making it more difficult for G1 to reduce the level of the antisense transcript and block action of the pdRNA.

To investigate the potential for physical interactions between pdRNAs and antisense transcripts we modified the 3′ termini of pdRNAs strands with biotin. We tested RNA duplexes with biotin attached to either strand. We first demonstrated that biotin labeling did not affect activity. Biotinylated pdRNAs activated PR expression in MCF7 cells and inhibited PR expression in T47D cells with efficiencies similar to those shown by analogous unmodified pdRNAs.

We harvested cells, purified biotinylated material using beads modified with streptavidin, eluted bound material from the beads, and amplified it by qPCR. We detected the antisense transcript AT2 after transfecting MCF7 cells with activating pdRNA PR1110 biotinylated on the strand complementary to AT2. Similarly, antisense transcript AT2 could be purified after transfecting T47D cells with inhibitory pdRNA PR9^{3,6} biotinylated on the strand complementary to AT2. The identity of the amplified products was verified by sequencing.

Control experiments confirm that pdRNAs are binding to antisense transcripts. The antisense transcript was not detected after treatment with pdRNAs that lacked biotin. No amplified product was observed when the biotinylated strand was not complementary AT2 demonstrating the specificity of recognition. When we used primers designed to detect genomic DNA, no product was detected. This result indicates that there is no direct interaction between biotinylated pdRNAs and chromosomal DNA.

Grewal²², Eglin²², and Moazed²³ have described models for how transcribed RNA can act as a scaffold for protein complexes that affect heterochromatin formation in s. Pombe and d. Melanogaster. We hypothesized that antisense transcripts might also be acting as scaffolds for organizing proteins at promoters and reasoned that argonaute proteins would likely be involved.

To detect involvement of argonaute proteins in pdRNA-mediated activation of PR we performed chromatin immunoprecipitation (ChIP) using a well-characterized antibody capable of detecting all four of the argonaute proteins in human cells²⁴. Activation or inhibition of PR by pdRNAs was verified by western analysis of treated cells. ChIP followed by qPCR revealed a 5-fold greater association of argonaute protein with the promoter of PR in MCF7 cells treated with activating pdRNA PR11 relative to those treated with mismatched RNAs. We observed a similar increase in argonaute association with the PR promoter in T47D cells treated with silencing RNA PR9. These data demonstrate that activating and inhibitory pdRNAs recruit argonaute to gene promoters.

To investigate the potential for interactions between antisense transcript AT-2 and argonaute proteins we employed RNA immunoprecipitation (RIP)²⁵. This method is similar to chromatin immunoprecipitation but has been modified to detect RNA associated with proteins. We used the same anti-argonaute antibody²⁴ used in ChIP experiments described above.

We added activating RNA PR11 to MCF7 cells and then performed RIP. qPCR amplification revealed that addition of pdRNA PR11 promoted association of argonaute protein with antisense transcript AT2-MCF7. Little or no PCR product was observed upon addition of mismatch-containing duplex RNA or when a control IgG was used. We performed RIP using T47D cells and observed that silencing pdRNA PR9 also promoted association of argonaute to antisense transcript AT2-T47D.

Our data demonstrate that addition of pdRNAs to cells promotes association of argonaute, antisense transcripts, and chromosomal DNA at the PR promoter. Data with biotinylated pdRNAs indicate a direct interaction between pdRNAs and the antisense transcripts. The association between argonaute and chromosomal DNA is probably mediated through proteins, making it less direct. Our ChIP protocol includes a step that crosslinks protein and nucleic acid. It is likely that crosslinked proteins provide a bridge between argonaute proteins, the antisense transcript, and chromosomal DNA.

We hypothesized that formation of RNA/protein complexes at promoters would include interaction with RNA binding proteins other than argonaute. We chose to examine the potential role of heterogeneous ribonuclear protein-k (hnRNP-k) in the action of pdRNAs. hnRNP-k is a transcription factor that is prevalent in the nucleus and recognizes both RNA and DNA. It is involved in gene transcription, elongation, splicing, DNA repair and interacts with proteins that modify histones²⁶. The PR promoter contains potential binding sites for hnRNP-k, providing another reason to test its involvement.

We used ChIP with an anti-hnRNP-k antibody to characterize association of hnRNPk at the PR promoter. Transfection of cells with activating pdRNA PR11 or inhibitory pdRNA PR9 reduced levels of hnRNP-k at the PR promoter relative to addition of mismatch-containing RNA duplexes.

We then used RIP to determine whether hnRNP-k associates with PR antisense transcripts upon addition of pdRNAs. We performed RIP experiments using an anti-hnRNP-k antibody in MCF7 cells treated with activating pdRNA PR11 or in T47D cells treated with inhibitory pdRNA PR9. We observed that addition of either pdRNA PR11 or pdRNA PR9 enhanced association of hnRNP-k with the antisense transcript AT2. Taken together, the ChIP and RIP data demonstrate that addition of pdRNAs shifts localization of hnRNP-k from chromosomal DNA to the antisense transcript, indicating that pdRNAs can induce the remodeling of protein interactions at gene promoters.

The ability of pdRNAs to activate or inhibit gene expression has been controversial²⁷, in part because the pdRNAs had no clear molecular target. Recently, Morris and coworkers have reported association of a pdRNA that inhibits expression of elongation factor 1α(EF1a) with a sense transcript that originates upstream of the EF1a transcription start²⁸. By contrast, we observe the following: i) binding of pdRNAs to antisense transcripts that originate within the target gene ii) interactions with the antisense transcript can lead to gene activation as well as gene silencing, and iii) pdRNAs can recruit proteins to antisense transcripts and shift the localization of proteins from promoter DNA. Our data show dynamic associations between antisense transcripts, promoter DNA, argonaute proteins, and hnRNP-k.

Like protein factors and small molecules, pdRNAs can activate gene expression in one cellular context and inhibit it in another. In both MCF7 and T47D cell lines, expression levels are poised to change upon addition of small molecule ligands or by altering cell culture conditions. For example, addition of estrogen will increase PR expression in MCF7 cells29, while removal of hormone-like compounds will reduce PR expression in T47D cells30. Small molecules alter expression by changing the recruitment of proteins at the promoter. If these small molecules can remodel the protein machinery at the PR promoter and affect RNA and protein synthesis, it should not be surprising that RNA-mediated recruitment of proteins can also trigger or repress gene expression.

We disclose a model for pdRNA-mediated modulation of gene expression. After entering cells, pdRNAs complementary to the PR promoter form a complex with argonaute protein and recognize an antisense RNA transcript. The antisense transcript:pdRNA:argonaute complex then acts as a scaffold for recruiting or redirecting other factors, such as hnRNP-k. This pdRNA:antisense RNA transcript:protein complex forms in proximity to the promoter, affecting the balance of regulation. For MCF7 cells, which are already poised to be induced for higher expression, the balance is pushed towards activation of PR expression. For T47D cells, the balance is pushed towards gene silencing.

We have extended our protocol to demonstrate modulation of diverse target gene expression using gapmers targeting antisense transcripts. Each of the following are sense antisense pairs overlapping the 5′ ends of genes. The gene is written out with its refseq number (NM_******) and is searchable in the Nucleotide database at Pubmed. After that is the sequence of the antisense transcript also searchable in the same database. After that is a table of gapmers which effectively modulate target gene expression by targeting the antisense transcript.

1. Tumor Suppressor Candidate Protein 4 (NM_{—}006545)

Antisense Transcript sequence AL552018

(SEQ ID NO:83)

GGGATGACGTCGCACCCGGAANTAAAGCSGCTCCGTGACGGAGCGGCGGT
GCGCGCGGCAGGGCCCGGAGTATCCCGCTTTCTTTGGAGGAAACAACCGC
ATCAGATCTGCGCTGCGGCAGAGGCAGGCAAGTCCCTAGCGTGGAGGGGC
AGCATGCTGGCAGCACTTGGGGAGGCGGTGCGCTAAGGGATTCACGCTGT
AACTGGGACCGCAGCAGGGAACTACAATTTCCATAGTGCTCCGCGCCCTC
CCAGCTGGCTCTACTGCCGGCGACGGCGTGGTACACTGGGATTTGTAGTC
TTACATGGCTTTGCGCATCCTACCTGGAAGGCGGGCCAGCGATTGGTACC
AGTTCAGACATGGGTACACGTTGACAGGCCGCCGGCGTCGACTGGCATGT
TGTGACCATTCCTGGTGCTGGTCTTGGTACTGTTCTTTCCTACCATAACT
TATTGGAAGAGGGTGGCATTCCTGCCTTGCAGCCTTTTCTCCAGTGAGGA
GTGAACAGTGGGCACCTGAGATCCTGGCCCACGCTACTATGCTTTCAGGC
TACAACCACTAGCACGGCTGACGATGGCCCTTTCTGCGGAGACCGAGTCA
CACATCTACCGAGCTCTGCGTACTGCTTCTGGCGCTGCCGCCCACCTTGT
GGCCCTGGGCTTTACCATCTTTGTGGCTGTGCTTGCCAGGCCTGGCTCCA
GCCTGTTCTCCTGGCACCCGGTGCTTATGTCTTTGGCTTTCTCCTTCCTG
ATGACCGAGGCACTACTGGTGTTTTCTCCTGAGAGTTCGCTGCTGCACTC
CCTCTCACGGAAAGGCCGAGCACGCTGCCACTGGGTGCTGCAGCTGCTGG
CCCTGCTGTGTGCACTGCTGGGCCTCGGCCTTGTCATCCTCCACAAAGAG
CAGCTTGGCAAACCCACCTGGTTACGCGGCATGGGCAGGCAGGGYKCTGG
CTGTTCTGTGGGCARGGCTGCAKTCTCAGGTGGGGTGGGGCKSYCYACCC
MAGCTGYKCCCSATGGCCCCTGCGAATCAAASTWWCCATGTACYTYTRGG
GTGKGGGYACYBCTGGGAATCCACCYYTTCTGG
Gapmers: Underlined bases are modified; the other
bases are DNA.

| | | |
|---|---|---|
| TUSC-13 | 5′ GCGGTCCCAGTTACAGCGT 3′ | (SEQ ID NO:84) |
| TUSC-50 | 5′ GGGCGCGGAGCACTATGGA 3′ | (SEQ ID NO:85) |
| TUSC-190 | 5′ GCCGGCGGCCTGTCAACGT 3′ | (SEQ ID NO:86) |
| TUSC-150 | 5′ CCAATCGCTGGCCCGCCTT 3′ | (SEQ ID NO:87) |
| TUSC-140 | 5′ CAGGTAGGAGGCGCAAAGC 3′ | (SEQ ID NO:88) |
| TUSC-500 | 5′ CCAGGCCTGGCAAGCACAG 3′ | (SEQ ID NO:89) |
| TUSC-380 | 5′ ATCGTCAGCCGTGCTAGTG 3′ | (SEQ ID NO:90) |
| TUSC-230 | 5′ AAGACCAGCACCAGGAATG 3′ | (SEQ ID NO:91) |
| TUSC-977 | 5′ GCACCGGGTGCCAGGAGAA 3′ | (SEQ ID NO:92) |
| TUSC-973 | 5′ CGGGTGCCAGGAGAACAGG 3′ | (SEQ ID NO:93) |

2. Leucine Zipper putative tumor suppressor 1 (NM_{—}021020)

| Antisense Transcript sequence BC033138 | |
|---|---|
| (SEQ ID NO:94) | |
| 1 | gctgatggaa gggaggtcag cccacagcct ggctgggcct tggtcatctg gcttccggct |
| 61 | tcatgattta atggctcact tgggaaactg aaatctagga gccatgaggg tgatggtggg |
| 121 | gacaggagga agctcagatg taagtcgatc ccccaacatg gtttgcaggg agccccttct |
| 181 | ttgggtgata aagccagcac attagccccg cttgcctgcg cggtctgtgt ttgcacgcta |
| 241 | ttggccggca ccagaaggag aggggggtac tggcgccaaa ccgctgacca cccaaaccca |
| 301 | tgagccctgt gtggcctcac ctcccactgg gtctcctcca gcgcggggcc gaagctggtc |
| 361 | ttctccctct cgtaggactt gagcttgttg ccgcctttgg gctccgggcc ctccagctcg |
| 421 | tccctgcagc gccgcggccg ctcctcgtag gccaggctgg aggcaagctc cttctcctca |
| 481 | aagctgcgct gcagcttctg gagggcgccc tccctctcca acagcttctg ctccagctcc |
| 541 | tggatgctgc actcgtccgt ggagatgggg gagcggacac acgaggggcc cttgtctgcc |
| 601 | ttgttcgagt ggcccagctt gctacctccg tcggagaagg acagagcctt caggctcatc |
| 661 | atgttgctgt cctggaggac gatgccctgg gtgatgttgt gggcggagcc cccaaaacgg |
| 721 | cttgtgggtc ccacgggtgt gaccagcggg tccagctggt agctgctgct ggtgctgtgt |
| 781 | gtgggcaggc tggacatgga gttccggccg gagtctgaca gcgccccaga gcacaggcca |
| 841 | ggcttcagct cctgctcctt gggcttgtct ggaggggcgg ggtgcagctg gtggctggca |
| 901 | ctctccgggg aggagtgcag gatggctcct gaccgtggca gcacaggctt gaaggctgtg |
| 961 | ggcctcactg cacccttctc ggagccctgt agaggaaaag gaccgcggtg actcatgcct |
| 1021 | cccctgcgcg cgcattgcac cctccctccc caggcacgcg tgccgacctt gagccagtct |
| 1081 | gggctctctg agcgcacgca gcacccctct tggtcaattg tctcagcaga ccttgcctgt |
| 1141 | tgctttgaag cagctgaatg tcatctctct taggaaggaa aaaccctaat ggcgacttgg |
| 1201 | gcactttgtt ctatgaaata gcaacctgcc accagcttgc cccagccctc ccgaggtgat |
| 1261 | aaataccatc ttgaggctcc tgctctaggt ctctgtgtgg ggcaagttag ggcatcaggc |
| 1321 | tggccgagct tgctgtccct ctttaggtcc atcccttctt cctgttcact acctcttcca |
| 1381 | ttaagcctgg agcaaggaca cggacctggc ctccttacag ggttgggagg ctcactccaa |
| 1441 | atcacgatcc ttttttaaaa ctgtaatttt ccctggtaga gtgcttcaca gtttacaagc |
| 1501 | cccttaattt gaaaagctga atgctctatg caaacataag aggctctttc ctagtaaatc |
| 1561 | aaagccgggg attcatttcc ccagggcaag gggcagagga ctgaatagga aaattgattt |
| 1621 | cagtgtccac tcgtgggacg cacgagggct tgagctggtg tgagggctgg atttctcagt |
| 1681 | gcctgggcct cctttgccct aatctctggt aaatggatga caaaactcca gcctgtattc |
| 1741 | aaagatgccc ccaggcgcag cttgaacaag gagctaatgc acaccagggc agcaaatgag |
| 1801 | aagaccgcac ccacccccac gagtctcccg gggagagaag cggttaactc ccggcctgca |
| 1861 | tcctcttcat ctgtgcttcc agatgagaac agggctccct ctccttcccg aggcttggca |
| 1921 | aacgcctgga tcctacgttg acaatccagc tacatttcag tgggactcca gaaagctcac |
| 1981 | atatcccctg tgctctttgc ttatggcctg acccaagact tctgcttcag ggggactgag |
| 2041 | cgatgctcta attcctttgt gaaacgtttg atctctgcgg tgtggccaca ggcttccgcc |
| 2101 | ggcacccctg ccgctctggt tttgaggagt ctgaatgctc aggtcaccac tccccctgaa |
| 2161 | cccccaggct ctccaccccc attttgcttt ctcctgcgtt tccaacccac ttacccttgc |
| 2221 | cagcgacccc cgcttaccat ttctagctga ttggagaagg gcatgagctt ggggggtgtg |
| 2281 | gacgggtcaa agtccacccc agcctggccc cctaaatccc cgctggacag tgccgtgtaa |
| 2341 | tctgggtgat gggagccccg ggctttctgg ctgaccttga tgtagaagaa gtcttcgctc |
| 2401 | ttgcccattt tggagctgga cttgccgtga ccggagtcct gggagaagcc aaacctcagc |
| 2461 | agcccgtcgg aataccggtt gagcttcttg aggtgggagg acttgcgcag cttgtactgc |
| 2521 | gaagcccggc agtgcttgct gtggaagctg tggccggaga tgaggctact gacgctgccc |
| 2581 | atggtgactc ggggctgagg atggggcagg gccgggcagg gtcttggaaa ggctgtggca |
| 2641 | gcaaggggca gtcgtggctc cgtgagggga ctgaggtcat agcaaagccc tcacagagcc |
| 2701 | tgcgagagcc gtagacctgg aagaagacac aagacagaag tcagcgtggg tgggagtggt |
| 2761 | cacagcacag tgctgattcc gacttccaga tctgggcagt agagaaacac atgtcagagc |
| 2821 | ccagcactgt ctccaccacc gggtgctccg ttccttcttc ctttgtctga taattcaaca |
| 2881 | aacattcact gggagcccac ttcatgcaag cctctactct aaacactcgg gacccagcaa |
| 2941 | gtgactaaaa cagtcacagt ccctgcattc ctggaactta gggttacagt ggtgtggaga |
| 3001 | ggccagaagc aaaccaatgt atactatagc aagtggtacc agtgctgcgg gtaccagtgc |
| 3061 | tgcgggtacc agtgctgcgg gggaaactgg agcagagagg acagaacaga attctgggag |
| 3121 | gtggctgttt tatgcaggaa ggtcttcttt tgttagggta gcattttaaa aggcttcaag |
| 3181 | aaaatgaggg ggcagccagg catgatggtt cacacctgta atcccagcac tctgggaggc |
| 3241 | tgaggtgggc agattgcttg agtccaggag ttcgagacca gcctgtgcaa cttagagaaa |
| 3301 | ccccatttct actaaaaata caaaaattag ccgggcgtag tggtgcacac ctgtaatccc |
| 3361 | agctacttgg gagggtgagg tgggagaatc gtctgagccc cggggatcaa ggctgcagtg |
| 3421 | agccacaatt gtgccactgc actccagcct gggcctgact caaaaaaaaa aaaaaaaaa |

| | | |
|---|---|---|
| Gapmers: | | |
| LZTS1-9 | 5′ CAGGCTCTGTGAGGGCTTT 3′ | (SEQ ID NO:95) |
| LZTS1-52 | 5′ CGCTGACTTCTGTCTTGTG 3′ | (SEQ ID NO:96) |
| LZTS1-150 | 5′ CCCGGTGGTGGAGACAGTG 3′ | (SEQ ID NO:97) |
| LZTS1-690 | 5′ CCACCTCACCCTCCCAAGT 3′ | (SEQ ID NO:98) |
| LZTS1-550 | 5′ TCAGCCTCCCAGAGTGCTG 3′ | (SEQ ID NO:99) |
| LZTS1-400 | 5′ GCTCCAGTTTCCCCCGCAG 3′ | (SEQ ID NO:100) |
| LZTS1-250 | 5′ CACTTGCTGGGTCCCGAGT 3′ | (SEQ ID NO:101) |
| LZYS1-140 | 5′ GAGACAGTGCTGGGCTCTG 3′ | (SEQ ID NO:102) |

3. Tumor protein p53 (NM_{—}000546)

| Antisense transcript sequence AK056669 | |
|---|---|
| (SEQ ID NO:103) | |
| 1 | agaaatgtaa atgtggagcc aaacaataac agggctgccg ggcctctcag attgcgacgg |
| 61 | tcctcctcgg cctggcgggc aaacccctgg tttagcactt ctcacttcca cgactgacag |
| 121 | ccttcaattg gattttctcc atctagcgga gccgggggct gcctggaaag atcgctccag |
| 181 | gaaggacaaa ggtccggaag ttgtgggacc ttagcagctt gggctccccg gatcaccccc |
| 241 | aaatgatcat ttcggaatgg agccccagtt ttcactagga tgccatgggc tctaaaatat |
| 301 | acagctatga gttctcaatg tttcgagatc caaaagtctc agacctcaat gctttgtgca |
| 361 | tcttttattt caaggattcc ctacgcccag caccgggtgg atgtgcaaag aagtacgctt |
| 421 | taggccggct caaggttccc caaagctcca ctcctctgcc taggcgttca actttgagtt |
| 481 | cggatggtcc taacatcccc atcatctaca cccaggtctc ccaacaatgc aactcctatg |
| 541 | atgatccctc tagccaagct tccatcccac tcacccccaa actcgctaag tccccactgc |
| 601 | cccaccccca gccccagcga ttttcccgag ctgaaaatac acggagccga gagcccgtga |
| 661 | ctcagagagg actcatcaag ttcagtcagg agcttaccca atccagggaa gcgtgtcacc |
| 721 | gtcgtggaaa gcacgctccc agcccgaacg caaagtgtcc ccggagccca gcagctacct |
| 781 | gctccctgga cggtggctct agacttttga gaagctcaaa acttttagcg ccagtcttga |
| 841 | gcacatggga ggggaaaacc ccaatcccat caacccctgc gaggctcctg gcacaaagct |
| 901 | ggacagtcgc catgacaagt aagggcaagt aatccgcctg ccggaggaag caaaggaaat |
| 961 | ggagttgggg aggagggtgc agagtcagga ttctcgccga cctggtgccg tagatactaa |
| 1021 | cattttgggg tggaaaattc tgcaagccag agctgtgagg gcagaattgg tggaaatcat |
| 1081 | tttggaggaa tcctgcattg tgtcaaatat gaagggtgga aggaagaaag cttttgcgtt |
| 1141 | tgctctcagc tggatccttt cttctcatca gttaaaatgt cattttttag gaaggctttc |
| 1201 | cgtaatatca caccctaacg ttttctccca gatactttat atcacaccat cttatttaat |
| 1261 | ctccttcaca acccttatca ctctgataag atttatttgt tcattgcttt cagtacatgg |
| 1321 | aaacgtaagc cttatgagga tatagaattt ttctactatc ttattcattg ttgtattcct |
| 1381 | gagtgcctat atcagtgctg ggtagcaagt aagagctcga taataaatat tttttgaatg |
| 1441 | agggagacag gtctgaagcc tggagaatga gatgcagaag aggtgcaaga cctgctgcgc |
| 1501 | cctctgcagg cggcgggggg gcggtgcagg tgctttaaga attaccgcgg gactcggtag |
| 1561 | ggggagcgta ggcgcttctc gccaagatag aagcgttcag actacaactc ccagcagcca |
| 1621 | cgaggagccc tagggcttga tgggaacggg aaaccttcta acctttcacg tcccggctcc |
| 1681 | gcgggttccg tgggtcgccc gcgaaatctg atccgggatg cggcggccca atcggaaggt |
| 1741 | ggaccgaaat cccgcgacag caagaggccc gtagcgaccc gcggtgctaa ggaacacagt |
| 1801 | gctttcaaaa gaattggcgt ccgctgttcg cctctcctcc cgggagtctt ctgcctactc |
| 1861 | ccagaagagg agggaagcac atgtgggttt ctttagctct gcgtcggatc cctgagaact |
| 1921 | tcgaagccat cctggctgag gctaatctcc gctgtgcttc ctctgcagta tgaagacttt |
| 1981 | ggagactcaa ccgttagctc cggactgctg tccttcagac caggacccag ctccagccca |
| 2041 | tccttctccc cacgcttccc cgatgaataa aaatgcggac tctgaactga tgccaccgcc |
| 2101 | tcccgaaagg ggggatccgc cccggttgtc cccagatcct gtggctggct cagctgtgtc |
| 2161 | ccaggagcta cgggaggggg acccagtttc tctctccact cccctggaaa cagagtttgg |
| 2221 | ttcccctagt gagttgagtc ctcgaatcga ggagcaagaa ctttctgaaa atacaagcct |
| 2281 | tcctgcagaa gaagcaaacg ggagcctttc tgaagaagaa gcgaacgggc cagagttggg |
| 2341 | gtctggaaaa gccatggaag atacctctgg ggaacccgct gcagaggacg agggagacac |
| 2401 | cgcttggaac tacagcttct cccagctgcc tcgatttctc agtggttcct ggtcagagtt |
| 2461 | cagcacccaa cctgagaact tcttgaaagg ctgtaagtgg gctcctgacg gttcctgcat |
| 2521 | cttgaccaat agtgctgata acatcttgcg aatttataac ctgcccccag agctgtacca |
| 2581 | tgagggggag caggtggaat atgcagaaat ggtccctgtc cttcgaatgg tggaaggtga |
| 2641 | taccatctat gattactgct ggtattctct gatgtcctca gcccagccag acacctccta |
| 2701 | cgtggccagc agcagccggg agaacccgat tcatatctgg gacgcattca ctggagagct |
| 2761 | ccgggcttcc tttcgcgcct acaaccacct ggatgagctg acggcagccc attcgctctg |
| 2821 | cttctccccg gatggctccc agctcttctg tggcttcaac cggactgtgc gtgttttttc |
| 2881 | cacggcccgg cctggccgag actgcgaggt ccgagccaca tttgcaaaaa agcagggcca |
| 2941 | gagcggcatc atctcctgca tagccttcag cccagcccag cccctctatg cctgtggctc |
| 3001 | ctacggccgc tccctgggtc tgtatgcctg ggatgatggc tcccctctcg ccttgctggg |
| 3061 | agggcaccaa gggggcatca cccacctctg ctttcatccc gatggcaacc gcttcttctc |
| 3121 | aggagcccgc aaggatgctg agctcctgtg ctgggatctc cggcagtctg gttacccact |
| 3181 | gtggtccctg ggtcgagagg tgaccaccaa tcagcgcatc tacttcgatc tggacccgac |
| 3241 | cgggcagttc ctagtgagtg gcagcacgag cggggctgtc tctgtgtggg acacggacgg |
| 3301 | gcctggcaat gatgggaagc cggagcccgt gttgagtttt ctgccccaga aggactgcac |
| 3361 | caatggcgtg agcctgcacc ctagcctgcc tctcctggcc actgcctccg gtcagcgtgt |
| 3421 | gtttcctgag cccacagaga gtggggacga aggagagggg ctgggccttc ccttgctctc |
| 3481 | cacgcgccac gtccaccttg aatgtcggct tcagctctgg tggtgtgggg gggcgccaga |
| 3541 | ctccggcatc cctgatgatc accagggcga gaaagggcag ggaggaacgg agggaggtgt |
| 3601 | gggtgagctg atataaaaag gtttttatg |

| | | |
|---|---|---|
| Gapmers: | | |
| TP53-18 | 5′ GGCGGATTACTTGCCCTTA 3′ | (SEQ ID NO:104) |
| TP53-50 | 5′ CCTCCCCAACTCCATTTCC 3′ | (SEQ ID NO:105) |
| TP53-182 | 5′ CACAATGCAGGATTCCTCC 3′ | (SEQ ID NO:106) |
| TP53-1480 | 5′ GTGTCTCCCTCGTCCTCTG 3′ | (SEQ ID NO:107) |
| TP53-1260 | 5′ CCCCCTCCCGTAGCTCCTG 3′ | (SEQ ID NO:108) |
| TP53-540 | 5′ GGCTTCAGACCTGTCTCCC 3′ | (SEQ ID NO:109) |
| TP53-140 | 5′ CCTCACAGCTCTGGCTTGC 3′ | (SEQ ID NO:110) |
| TP53-15649 | 5′ ACCCTAGCCTGCCTCTCCT 3′ | (SEQ ID NO:111) |

4. Progesterone Receptor (NM_000926)

Antisense transcript sequence—AT2-T47D; bold nucleotides indicate the beginning of each exon in the transcript AT2-T47D (Exon 1+536 to −71; Exon 2-871 to −964; Exon 3-3193 to −3309; Exon 4 −18327 to −18416; Exon 5 −29343 to −29440; Exon 6 −65672 to −65822; Exon 7 −68912 to −69083)

(SEQ ID NO:112)

AACTGTGGCTGTCGTTTGTCCCAGCGAGCGGCAAGTGGGGAGCGCAAGAA
AAAGTAGTAATTGTTAGGAGATCTCGTCTCCTAACTCGGGGAGTTCTCCA
AGAGAGTTCTCCAACTTCTGTCCGAGGACTGGAGACGCAGAGTACTCACA
AGTCCGGCACTTGAGTGGCTGCGGCTGCGACGGCAATTTAGTGACACGCG
GCTCCTTTATCTCCCGACTTTTTCTCTGGCATCAAACTCGTGCATGCTGT
GAAGCTCTCAGTCCCTCGCTGAGTTCCACTGCCCCCTCACTAAAACCCTG
GGGCTAGTCGGACCTCTCGGTACAGCCCATTCCCAGGAAGGGTCGGACTT
CTGCTGGCTCCGTACTGCGGGCGACAGTCATCTCCGAAGATCTCAGATCC
CAGTAGTGCGGGAGCACTAGCCGCCTCGGGTTGTAGATTTCACTCAAATG
ACAAGTGAAGCTAGTTCTCATTGAGAATGCCACCCACACGCACAAATACA
ACAAGGCTTACCCCGATTAGNGACAGNTGTGGACTNNCCAGACAGNTTTN
TAACAANGCCTCCTCNTCTAGGGNNGNCCCGCCCAAAGCCCCTCCCTACC
CCAATTACCGGACCTCAAGGTCTAGCTGTGCTAATGACTCATAGTTTATN
TCANCCATGTATAAAGAATGCAGAAGACTCCAGAAGGTGGGGGAGCCACT
AGAGGATTCCATCCAGGACACCACATTTAATTGTTATGATGTCTTGTGCT
CCTCTTGGCTGTGAGAGTTTCTCAGATTTTCCTTGTATTTGATGACCTTG
ACAGTCCTTAGGAGTACTGAGATGGGGGTTTCACGATATTGCCCAGGCTG
GTCCCAAACTCCTGGCCTCAAGCTATCCTCCTGCCTTGACGTTCGAAAGC
ACTNAGATTTTCCTNTCTGTCCTGCCGCCATGTGAAGAAANATGTGTTTG
CTTCCCCTTCCNCCGTGATTGTANTTTTCCTGCAGCCTCCCCAGCCAAGC
TGAACTGCAATGAGGAAGCAATAAAGGACTNTGAGCAAGAGAACGATGTA
TATAGGCCATTGTTTTGGAAGATTCATCNCAAATTCATGTGCAAGTAGAT
TGGAGGAAGGAAGTATCAACANAAANAAAGGCCAATTATGAGACCATTGC
AATACTATATGATGAGAGCATCAGAGNCAATAACCAAGGTNTTATAGATA
CCAAGTGAGGAATCTGGGATTANAAGTCAGTNTATTTCATTGGAAAGGCT
GTTTTCAGTNTTTTTCACTGGAAAAGTTGTCATCCTGTGTCTTTNTTATA
GTACATATATTNCAGTAATAAAACTTTATTTTCCTTTTCAAAAAAAAAAA
AAAAAAAAA

| | | |
|---|---|---|
| Gapmers: | | |
| PR-20 | TGTTAGAAAGCTGTCTGGCC | (SEQ ID NO:73) |
| PR-32 | GAGGAGGCGTTGTTAGAAAG | (SEQ ID NO:74) |
| PR-35 | TAGAGGAGGAGGCGTTGTTA | (SEQ ID NO:75) |
| PR-3195 | GTGGTGTCCTGGATGGAATC | (SEQ ID NO:113) |
| PR-68951 | TCCCAGATTCCTCACTTGGT | (SEQ ID NO:114) |

### REFERENCES

1. Morris K. V., Chan S. W., Jacobsen S. E. & Looney D. J. Small interfering RNA-induced transcriptional silencing in human cells. Science 305, 1289-92 (2004).
2. Ting, A. H., Schuebel, K. E., Herman, J. G. & Baylin, S. B. Short double-stranded RNA induces transcriptional gene silencing in human cells in the absence of DNA methylation. Nat. Genetics 37, 906-910 (2005).
3. Janowski, B. A. et al. Inhibition of gene expression at transcription start sites using antigene RNAs (pdRNAs). Nat. Chem. Biol. 1, 216-222 (2005).
4. Suzuki K. et al. Prolonged transcriptional silencing and CpG methylation induced by siRNAs targeted to the HIV-1 promoter region. J. RNAi Gene Silencing 1, 66-78 (2005).
5. Zhang M-X. et al. Regulation of endothelial nitric oxide synthase by small RNA. Proc. Natl. Acad. Sci USA 102, 16967-16972 (2005).
6. Janowski, B. A. et al. Involvement of Ago 1 and Ago2 in mammalian transcriptional silencing. Nat. Struc. Mol. Biol. 13, 787-792 (2006).
7. Kim, D. H. et al. Argonaute-1 directs siRNA-mediated transcriptional gene silencing in human cells. Nat. Struc. Mol. Biol. 13, 792-797 (2006).
8. Pulukuri, S. & Rao, J. S. Small interfering RNA-directed reversal of urokinase plasminogen activator dimethylation inhibits prostate tumor growth. Cancer Res. 67, 6637-6646 (2007).
9. Li, L. et al. Small dsRNAs induce transcriptional activation in human cells. Proc. Natl. Acad. Sci. USA 103, 17337-17342 (2006).
10. Janowski, B. A. et al. Activating gene expression in mammalian cells with promoter-targeted duplex RNAs. Nat. Chem. Biol. 3 166-173 (2007).
11. The FANTOM Consortium. The transcriptional landscape of the mammalian genome. Science 309, 1559-1563 (2005).
12. RIKEN Group and FANTOM Consortium Antisense transcription in the mammalian transcriptome. Science 309, 1564-1566 (2005).
13. The Encode Project Consortium. Identification and analysis of functional elements in 1% of the human genome by the Encode pilot project. Nature 447, 799-816 (2007).
14. Gingeras, T. R. Origin of phenotypes: genes and transcripts. Genome Res. 17, 682-690 (2007).
15. Meister, G. et al. Human argonaute2 mediates RNA cleavage targeted by miRNAs and siRNAs. Mol. Cell. 15, 185-197 (2004).
16. Liu, J. et al. Argonaute2 is the catalytic engine of mammalian RNAi. Science 305, 1437 (2004).
17. Invitrogen. Advanced RACE Method Amplifies Only Full-Length cDNA Ends. Expressions 7.3, 2-3 (2000).
18. Kastner, P. et al. Two distinct estrogen-regulated promoters generate transcripts encoding the two functionally different human progesterone receptor forms A and B. EMBO J. 9, 1603-14 (1990).
19. Misrahi, M. et al. Structure of the human progesterone receptor gene. Biochim. Biophys. Acta 1216, 289-92, (1993).
20. Zhang, H. et al. Reduction of liver Fad expression by an antisense oligonucleotide protects mice from fulminant hepatitis. Nature Biotech. 18, 862-867 (2000).
21. Corey, D. R. RNAi learns from antisense. Nat. Chem. Biol. 3, 8-11 (2007).
22. Grewal, S. I. S. & Eglin, S. C. R. Transcription and RNA interference in the formation of heterochromatin. Nature 447, 399-406 (2007).
23. Buhler, M., Verdel, A., & Moazed, D. Tethering RITS to a nascent transcript initiates RNAi-and heterochromatin dependent gene silencing. Cell 125, 873-886 (2006).
24. Nelson, P. T. et al. A novel monoclonal antibody against human argonaute proteins reveals unexpected characteristics of miRNAs in human blood cells. RNA 13, 1787-1792 (2007).
25. Gilbert C., Kristjuhan, A., Winkler G. S. & Svejstrup J. Q. (2004). Elongator interactions with nascent mRNA revealed by RNA immunoprecipitation. Mol. Cell. 14, 457-64 (2004).
26. Bomsztyk, K., Denisenko, O. & Ostrowski, J. (2004). hnRNP-k: One protein multiple processes. Bioessays 26.6., 629-638 (2004).
27. Check, E. RNA interference: hitting the switch. Nature 448, 855-858 (2007).
28. Han, J., Kim, D. & Morris, K. V. Promoter-associated RNA is required for RNA-directed transcriptional gene silencing in human cells. Proc. Natl. Acad. USA 104, 12422-12427 (2007).
29. Lee, Y. & Gorski, J. Estrogen-induced transcription of the progesterone receptor gene does not parallel estrogen receptor occupancy. Proc. Natl. Acad. Sci USA 93, 15180-15184 (1996).
30. Hurd, C. et al. Hormonal regulation of the p53 tumor suppressor protein in T47D human breast carcinoma cell line. J. Biol. Chem. 270, 28507-28510 (1995).

The foregoing description and examples are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of selectively modulating expression of a target gene in the genome of a mammalian cell determined to be in need thereof, comprising:
determining the presence of an encoded antisense transcript overlapping a promoter of the target gene;
contacting the transcript with an exogenous gapmer or double-stranded agRNA; and
detecting a resultant modulation of expression of the target gene, the gapmer comprising a DNA insert complementary to a portion of the transcript upstream relative to the transcription start site of the gene, and the agRNA being 18-28 bases and complementary to a portion of the transcript upstream relative to the transcription start site of the gene.

2. The method of claim 1 wherein the determining step is implemented in silico by examining transcriptional data to identity the antisense transcript.

3. The method of claim 1 wherein the determining step is implemented in vitro by using 5′-RACE/3′-RACE to experimentally identify the antisense transcript.

4. The method of claim 1 wherein the agRNA or DNA insert is complementary to a portion of the transcript more than 100 bases upstream relative to the transcription start site of the gene.

5. The method of claim 1 wherein the agRNA or DNA insert is complementary to a portion of the transcript more than 200 bases upstream relative to the transcription start site of the gene.

6. The method of claim 1 wherein the agRNA or DNA insert is complementary to a portion of the transcript more than 1,000 bases upstream relative to the transcription start site of the gene.

7. The method of claim 1 wherein the agRNA or DNA insert is a priori not known to be a modulator of the target gene.

8. The method of claim 1 wherein the antisense transcript is a priori not known to overlap the promoter of the target gene.

9. The method of claim 1 wherein the modulation is methylase-independent.

10. The method of claim 1 further comprising the step of confirming that the modulation is methylase-independent.

11. The method of claim 1 wherein the agRNA or DNA insert is complementary to a portion of the transcript outside a CpG island.

12. The method of claim 1 further comprising the step of confirming that the agRNA or DNA insert is complementary to a portion of the transcript outside a CpG island.

13. The method of claim 1 wherein the contacting step is free of viral transduction.

14. The method of claim 1 wherein the contacting step is implemented by contacting the cell with a composition consisting essentially of the agRNA or DNA insert.

15. The method of claim 1 wherein the contacting step is implemented by contacting the cell with a composition comprising the agRNA or DNA insert at 1-100 nanomolar concentration.

16. The method of claim 1 wherein the detecting step is implemented by detecting at least a 50% increased expression of the target gene.

17. The method of claim 1 wherein the detecting step is implemented by detecting at least a 200% increased expression of the target gene.

18. The method of claim 1 wherein the detecting step is implemented by detecting at least a 50% decreased expression of the target gene.

19. The method of claim 1 wherein the detecting step is implemented by detecting at least a 75% decreased expression of the target gene.

20. The method of claim 1 wherein no more than one portion of the transcript is targeted.
